(19) United States
(12) Patent Application Publication
(10) Pub. No.: US 2010/0113372 A1
(43) Pub. Date: May 6, 2010

(54) Method For Preparing Kaempferol-3-0-Rutinoside and Composition of Skin External Application Comprising Thereof

(75) Inventors: Jun Seong Park, Suwon-si (KR); Hye Yoon Park, Anyang-si (KR); Ho Sik Rho, Yongin-si (KR); Duck Hee Kim, Seoul (KR); Ih Seop Chang, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/445,362

(22) PCT Filed: Apr. 25, 2007
(86) PCT No.: PCT/KR07/02030

§ 371 Date: Jun. 17, 2009

(30) Foreign Application Priority Data
Oct. 13, 2006 (KR) 10-2006-0099725

(51) Int. Cl. A61K 31/70; C12P 19/60

(52) U.S. Cl. 514/27; 435/75

(57) ABSTRACT

Disclosed is a method for the preparation of kaempferol-3-O-rutinoside and a composition of a skin external application comprising kaempferol-3-O-rutinoside as an active ingredient. The method for isolating kaempferol-3-O-rutinoside through hydrolysis uses an enzyme or microbe that removes the sugar selectively from kaempferol-3-O-rutinoside glycosides in a plant extract. Disclosed also is a composition of a skin external application comprising kaempferol-3-O-rutinoside that prevents skin wrinkle.

### TECHNICAL FIELD

The present invention relates to a method for the preparation of kaempferol-3-O-rutinoside having the following chemical formula 1 and a composition of a skin external application comprising kaempferol-3-O-rutinoside as an active ingredient. In particular, the present invention relates to a method for isolating kaempferol-3-O-rutinoside through hydrolysis using an enzyme or microbe that removes the sugar selectively from kaempferol-3-O-rutinoside glycosides in a plant extract, and a composition of a skin external application comprising kaempferol-3-O-rutinoside that prevents skin wrinkle.

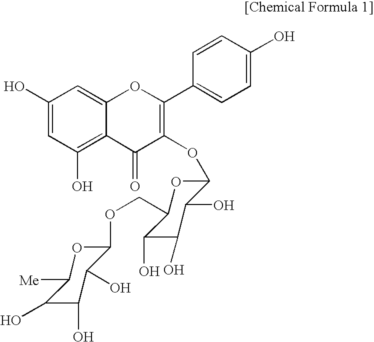

### BACKGROUND ART

Kaempferol-3-O-rutinoside having the above chemical formula 1 is a representative ingredient of flavonol, which is a flavonoid and is widely distributed in a flower and leaf of a plant (Redox report, 4, 13-16, 1999). In particular, kaempferol-3-O-rutinoside is a substance having excellent physiological activities, such as anti-oxidation (Redox Report, Vol. 4, No. 3, 1999) and improvement of blood circulation (Biol. Pharm. Bull. 25(4) 505-508, 2002). Accordingly, research on the various efficacies of kaempferol-3-O-rutinoside has been performed and kaempferol-3-O-rutinoside has been applied to diverse fields. However, since the kaempferol-3-O-rutinoside that is currently used is mostly contained in a plant extract in an amount of only several ppm to several tens of ppm, a substantial efficacy of kaempferol-3-O-rutinoside is difficult to reveal. In addition, since a plant containing a large quantity of kaempferol-3-O-rutinoside is difficult to find and there are no economical merits of isolation and purification for preparing a large quantity of kaempferol-3-O-rutinoside, research on mass production of kaempferol-3-O-rutinoside has seldom been performed.

In the meantime, the external skin is comprised of extracellular matrix (ECM) components of dermis tissue and collagen that account for about 70˜80% based on the total of ECM. The skin wrinkle is formed by reduced generation or by destruction of collagen, caused by aging or UV light. Particularly, the expression of matrix metallo protease, such as collagenase, causes decomposition of collagen produced normally, whereby wrinkles are generated.

To inhibit the reduction of collagen, which is the origin of wrinkling, various materials have been developed and used. The retinoid materials, such as retinol, retinoic acid and the like, show an excellent effect of preventing wrinkle (Dermatology therapy, 1998, 16, 357-364) and composition comprising malt extract and the like is applied for controlling collagenase (Japan Patent No. 5,105,693). However, retinoid materials stimulate the skin even when only a small quantity of retinoid material is used. Also, materials obtained from natural products are used in a form of simple extract, and the effect of each ingredient is not revealed. Therefore, it is difficult to maintain and control the activity of the extract continuously.

### DISCLOSURE

### Technical Problem

The inventors found that a green tea seed, which is not used for a specific purpose, contains a large quantity of glycosides such as camelliaside A and camelliaside B. From this finding, the inventors developed a method for mass-producing kaempferol-3-O-rutinoside having an excellent physiological activity, and also confirmed that kaempferol-3-O-rutinoside has an excellent effect of preventing wrinkle.

Accordingly, the object of the present invention is to provide a method for mass-producing kaempferol-3-O-rutinoside of high purity to be applied as cosmetic and food ingredients, and to provide a composition of a skin external application comprising kaempferol-3-O-rutinoside with an excellent effect of preventing wrinkle.

### Technical Solution

In order to accomplish the object, the present invention provides a preparation method of kaempferol-3-O-rutinoside having the following chemical formula 1 comprising isolating kaempferol-3-O-rutinoside through hydrolysis using an enzyme or microbe that removes the sugar selectively from kaempferol-3-O-rutinoside glycosides in a plant extract.

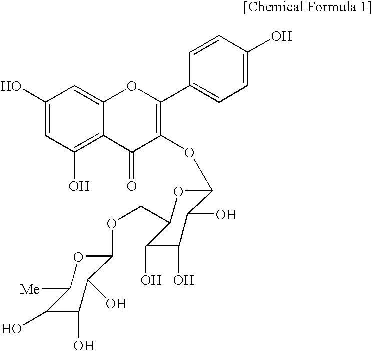

The present invention further provides a composition of a skin external application comprising kaempferol-3-O-rutinoside for preventing wrinkle.

Hereinafter, the present invention is described in detail.

The method for preparing kaempferol-3-O-rutinoside comprises:

(1) obtaining a plant extract containing kaempferol-3-O-rutinoside glycosides using one of water and organic solvent; and

(2) removing the sugar selectively from the kaempferol-3-O-rutinoside glycosides using one of an enzyme and a microbe in the extract to isolate kaempferol-3-O-rutinoside.

In order to obtain the plant extract containing kaempferol-3-O-rutinoside or kaempferol-3-O-rutinoside glycosides using water or an organic solvent from plants of step (1), specifically from green tea (Camellia sinensis), the water or organic solvent is poured to the green tea leaf or seed about one to six times, preferably about three times. The green tea leaf or seed is then extracted by stirring the mixture for one to five rotations at room temperature to remove fat. The water or organic solvent is poured to the fat-removed green tea leaf or seed about one to eight times, preferably about four times. The mixture is extracted under reflux one to five times, and deposited at 10° C. to 20° C. for one to three days. Residues and filtrate are then separated through filtration and centrifugation, extracts are obtained by concentrating the separated filtrate under reduced pressure, and the extracts are suspended in water and pigments of the extracts removed using the organic solvent. Next, the water layer is extracted one to five times using the organic solvent. An extract is obtained by concentrating the obtained organic solvent layer under reduced pressure, and then the extract is dissolved in a small quantity of the organic solvent. Precipitates are produced by adding a large quantity of the organic solvent to the mixture, and the precipitates are dried to obtain the extract containing kaempferol-3-O-rutinoside glycosides of the present invention.

The extract comprises kaempferol-3-O-rutinoside glycosides, specifically camelliaside A or camelliaside B.

The organic solvent may be at least one selected from a group consisting of ethanol, methanol, butanol, ether, ethylacetate and chloroform, and a mixture of the organic solvents and water, preferably 80% ethanol, may be used.

In step (2) of removing the sugar selectively from kaempferol-3-O-rutinoside glycosides using an enzyme or microbe in the extract to isolate kaempferol-3-O-rutinoside, the kaempferol-3-O-rutinoside is prepared from camelliaside A or camelliaside B, both of which are kaempferol-3-O-rutinoside glycosides, among the extract prepared in step (1) using an enzyme or microbe.

The enzyme decomposing a sugar bond is obtained from a microbe or the like. This enzyme may be a commercially marketed enzyme or may be prepared according to need. This enzyme particularly has an activity that removes the sugar selectively from the kaempferol-3-O-rutinoside glycosides to isolate kaempferol-3-O-rutinoside.

A reaction for preparing kaempferol-3-O-rutinoside from camelliaside A is shown in the following reaction formula 1.

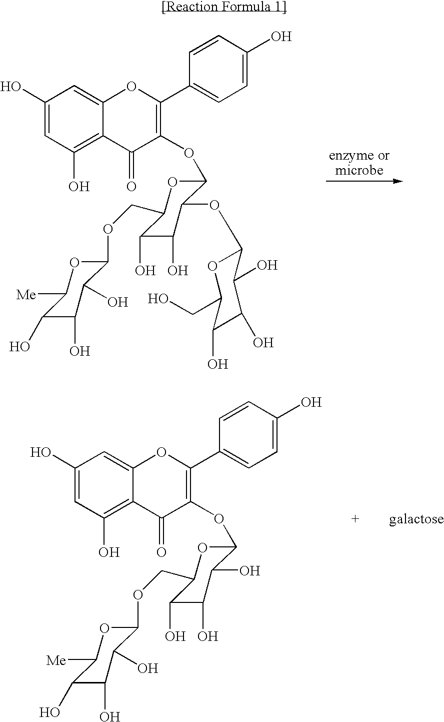

In the above reaction, kaempferol-3-O-rutinoside is obtained by removing selectively the sugar of galactopyranose group from camelliaside A. The enzyme for removing the sugar from camelliaside A may be at least one selected from a group consisting of glucosidase, cellulase, galactosidase and amyloglucosidase.

A reaction for preparing kaempferol-3-O-rutinoside from camelliaside B is shown in the following reaction formula 2.

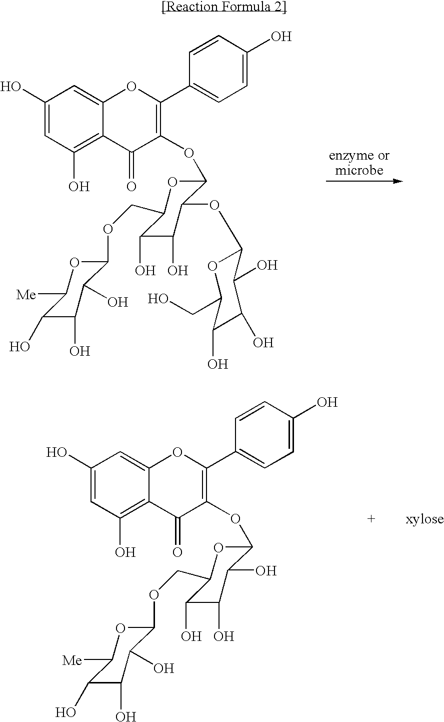

In the above reaction, kaempferol-3-O-rutinoside is obtained by removing selectively the sugar of xylopyranose group from camelliaside B. The enzyme for removing the sugar from camelliaside B may be at least one selected from a group consisting of xylosidase, xylanase and naringinase.

The microbe used in the reaction formulas 1 and 2 may be at least one selected from a group consisting of aspergillus sp., bacillus sp., penicillium sp., rhizopus sp., rhizomucor sp., talaromyces sp., bifidobacterium sp., mortierella sp., cryptococcus sp. and microbacterium sp.

When an enzyme or microbe is reacted, its pH range is preferably 4.0 to 5.5. If the pH is less than 4.0, the reaction rate is slow, and if the pH exceeds 5.5, the yield is low. Further, when the enzyme or microbe is reacted, the temperature range is preferably 30 to 50° C. If the temperature is less than 30° C., the reaction rate is slow, and if the temperature exceeds 50° C., the reaction selectivity of enzyme is decreased.

The concentration of the extract of green tea seed as the substrate is preferably in a range of 5 to 20%. If the concentration is greater than 20%, economical efficiency of the enzyme or microbe relative to the amount used decreases, and if the concentration is less than 5%, the reaction rate of the enzyme or microbe is low.

When the enzyme or microbe is reacted, the reaction time is preferably 48 to 76 hours.

A removal rate of the substrate is checked with a thin layer chromatography. When the substrate is completely removed, the mixture is heated in hot water (80-100° C.) for 5 to 15 minutes to terminate the reaction. The obtained reaction solution is concentrated under reduced pressure to remove the solvent, then alcohol is added to the residue and the mixture is stirred for one to five rotations. Precipitated salts are removed through filtration, and the filtrate is concentrated under reduced pressure to obtain crude products. The obtained crude products are purified with a silica gel column chromatography (chloroform: methanol=8:1˜4:1), thereby providing pure kaempferol-3-O-rutinoside.

The present invention provides a composition of a skin external application comprising kaempferol-3-O-rutinoside for preventing wrinkle.

The composition of a skin external application containing kaempferol-3-O-rutinoside obtained from a plant, in particular green tea, according to the said process has an excellent effect of preventing wrinkle caused by combined synergetic action of the promotion of procollagen generation and the inhibitory activity of collagenase expression.

Kaempferol-3-O-rutinoside is contained in an amount of 0.0001% to 10% by weight based on the total weight of the composition. If the kaempferol-3-O-rutinoside content is less than 0.0001% by weight, an effect of preventing wrinkle of the component cannot be obtained, and if the kaempferol-3-O-rutinoside content is greater than 10% by weight, increase of the effect is not remarkable as compared to the amount used.

Kaempferol-3-O-rutinoside of the present invention may be formulated into a composition of a skin external application, but is not particularly limited in its formulation. The formulation may include cosmetic compositions such as skin softener, nutrition water, nutrition cream, massage cream, essence, eye cream, eye essence, cleansing cream, cleansing foam, cleansing water, pack, powder, body lotion, body cream, body oil, body essence, makeup base, foundation, hair-dyeing agent, shampoo, rinse, body washing agent and the like; and pharmaceutical compositions such as ointment, gel, cream, patch, spraying agent and the like. In the composition of the respective formulations, various materials and additives needed for preparing the formulation may be added appropriately. The type and amount of the components are selected by the one skilled in the art without any difficulty.

### Advantageous Effects

According to the invention, camelliaside A or camelliaside B is extracted from a plant, particularly green tea, and then the sugar is removed selectively therefrom using an enzyme or microbe in order to mass-produce kaempferol-3-O-rutinoside, which is one of the main physiological active ingredients. The kaempferol-3-O-rutinoside exhibits the promotion of procollagen generation and the inhibitory activity of collagenase expression, so that it has an excellent effect of preventing wrinkle caused by combined synergetic action of the activities. The present invention provides a composition of a skin external application containing the kaempferol-3-O-rutinoside for preventing wrinkle.

### MODE FOR INVENTION

Hereinafter, the invention is more specifically described with a Preparation Example, Examples and Experimental Examples. However, the invention is not limited there to.

### Preparation Example 1

### Preparation of the Extract of Green Tea Seed

6 L of hexane were added to 2 kg of green tea seed and the mixture was stirred for three rotations at room temperature to remove fat from the extracted green tea seed. 4 L of 80% methanol were poured to 1 kg of the fat-removed seed, the mixture was extracted under reflux three times and the resulting solution deposited at 15° C. for one day. Residues and filtrate were separated through filtration by filter cloth and centrifugation. The separated filtrate was concentrated under reduced pressure and suspended in water, and then ether was added five times at a total of 1 L and extracted to remove pigments. 1-Butanol was added three times at a total of 500 ml and the water layer removed. The remaining 1-butanol layer obtained was concentrated under reduced pressure to obtain 1-butanol extract. The obtained extract was dissolved in a small quantity of methanol and then added to a large quantity of ethylacetate, thereby obtaining precipitates. The produced precipitates were dried, thereby obtaining 250 g of the extract of green tea seed.

### Example 1

### Selection of Enzyme Selectively Hydrolyzing Camelliaside A

10 g of the extract of green tea seed obtained from Preparation Example 1 was dissolved in 100 ml of 0.1M acetic acid buffer solution (pH 4.5). 1.5 g of enzyme was added to the mixture, and the resultant mixture was reacted in a water bath at 37° C. for 24 hours and 48 hours. Analysis of the conversion rate from camelliaside A was performed using High Performance Liquid Chromatography (HPLC) with C18 reverse phase column (mobile phase of acetonitrile: water=40:60) at a UV wavelength of 270 nm. The enzymes used in the experiment and the results of conversion rate are shown in the following Table 1.

TABLE 1

| | | Conversion rate (%) | |
|---|---|---|---|
| Enzyme | Origin | 24 hours | 48 hours |
| Amylase | Aspergillus oryzae | 12 | 32 |
| β-glucosidase | Almond | 44 | 65 |
| Amyloglucosidase | Aspergillus niger | 43 | 56 |
| Cellulase-T | Trichoderma reesei | — | — |
| Cellulase-A | Aspergillus niger | 34 | 65 |
| β-galactosidase | Aspergillus oryzae | 70 | 84 |
| β-xylosidase | Aspergillus niger | 13 | 23 |
| Xylanase | Trichoderma viride | 17 | 31 |
| Pectinase | Rhizopus sp | — | — |
| Naringinase | Penicillium decumbens | 7 | 12 |
| Hesperidinase | Aspergillus niger | 5 | 11 |

An enzyme that has about a 50% conversion rate after 48 hours is considered to be an enzyme with a high possibility of reaction selectivity in Table 1. Therefore, β-glucosidase, amyloglucosidase, cellulase-A, and β-galactosidase are included in a candidate group that has a high selectivity in the selective removal reaction of the sugar.

### Example 2

### Selection of Enzyme Selectively Hydrolyzing Camelliaside B

10 g of the extract of green tea seed obtained from Preparation Example 1 was dissolved in 100 ml of 0.1M acetic acid buffer solution (pH 4.5). 1.5 g of enzyme was added to the mixture, and the resultant mixture was reacted in a water bath at 37° C. for 24 hours and 48 hours. Analysis of the conversion rate from camelliaside B was performed using HPLC with C18 reverse phase column (mobile phase of acetonitrile: water=40:60) at a UV wavelength of 270 nm. The enzymes used in the experiment and the results of conversion rate are shown in the following Table 2.

TABLE 2

| | | Conversion rate (%) | |
|---|---|---|---|
| Enzyme | Origin | 24 hours | 48 hours |
| Amylase | Aspergillus oryzae | — | — |
| β-glucosidase | Almond | — | — |
| Amyloglucosidase | Aspergillus niger | — | — |
| Cellulase-T | Trichoderma reesei | 11 | 24 |
| Cellulase-A | Aspergillus niger | — | — |
| β-galactosidase | Aspergillus oryzae | — | — |
| β-xylosidase | Aspergillus niger | 64 | 77 |
| Xylanase | Trichoderma viride | 53 | 64 |
| Pectinase | Rhizopus sp | — | — |
| Naringinase | Penicillium decumbens | 48 | 69 |
| Hesperidinase | Aspergillus niger | 4 | 10 |

An enzyme that has about a 50% conversion rate after 48 hours is considered to be an enzyme with a high possibility of reaction selectivity in Table 2. Therefore, β-xylosidase, xylanase, and naringinase are included in a candidate group that has a high selectivity in the selective removal reaction of the sugar.

### Experimental Example 1

### Change of Conversion Rate of Kaempferol-3-O-Rutinoside According to Temperature

10 g of the extract of green tea seed obtained from Preparation Example 1 was dissolved in 100 ml of 0.1M acetic acid buffer solution (pH 4.5). 1.5 g of β-galactosidase from the candidate group was added to the mixture, and the conversion rate of kaempferol-3-O-rutinoside in a water bath at various temperatures was confirmed. The results of conversion rate are shown in the following Table 3.

TABLE 3

| Reaction temperature(° C.) | Conversion rate (%) |
|---|---|
| 25 | 79 |
| 30 | 92 |
| 35 | 98 |
| 40 | 97 |
| 45 | 94 |
| 50 | 91 |

It is confirmed that kaempferol-3-O-rutinoside is greatly converted at 30-50° C. In addition, the conversion rate at 35° C. is greater than the conversion rate at 30° C. and 40° C., which shows that the reactivity of enzyme increases according to temperature until 35° C. However, the conversion rate decreases significantly above 45° C., which results from instability of the enzyme according to temperature increase.

### Experimental Example 2

### Change of Conversion Rate of Kaempferol-3-O-Rutinoside According to Reaction Time

10 g of the extract of green tea seed obtained from Preparation Example 1 was dissolved in 100 ml of 0.1M acetic acid buffer solution (pH 4.5). 1.5 g of β-galactosidase from the candidate group was added to the mixture, and the conversion rate of kaempferol-3-O-rutinoside in a water bath at 37° C. for various reaction times was confirmed. The results of conversion rate are shown in the following Table 4.

TABLE 4

| Reaction time(hr) | Conversion rate (%) |
|---|---|
| 12 | 33 |
| 24 | 68 |
| 48 | 91 |
| 72 | 98 |
| 96 | 98 |
| 120 | 98 |

As shown in Table 4, a conversion rate of 98% is achieved after 72 hours and the conversion rate is similar above 72 hours.

### Experimental Example 3

### Change of Conversion Rate of Kaempferol-3-O-Rutinoside According to Reaction pH

10 g of the extract of green tea seed obtained from Preparation Example 1 was dissolved in 100 ml of 0.1M acetic acid buffer solution having various pH values. 1.5 g of β-galactosidase from the candidate group was added to the mixture, and the conversion rate of kaempferol-3-O-rutinoside in a water bath at 37° C. for various pH values of buffer solution was confirmed. The results of conversion rate are shown in the following Table 5.

TABLE 5

| Reaction pH | Conversion rate (%) |
|---|---|
| 4.0 | 93 |
| 4.5 | 98 |
| 5.0 | 95 |
| 5.5 | 90 |
| 6.0 | 84 |
| 6.5 | 77 |
| 7.0 | 75 |
| 7.5 | 78 |
| 8.0 | 80 |
| 8.5 | 82 |
| 9.0 | 83 |

As shown in Table 5, the conversion rate is greater than or equal to 90% for pH 4.0-5.5, and the greatest conversion rate is 98% for pH 4.5.

### Experimental Example 4

### Change of Conversion Rate of Kaempferol-3-O-Rutinoside According to Concentration of Substrate

The concentration of the extract of green tea seed obtained from Preparation

Example 1 was adjusted in the range of 5 to 50%. 1.5 g of β-galactosidase from the candidate group was added to the mixture, and the conversion rate of kaempferol-3-O-rutinoside in a water bath at 37° C. for various substrate concentrations was confirmed. The results of conversion rate are shown in the following Table 6.

TABLE 6

| Concentration of substrate (%) | Conversion rate (%) |
|---|---|
| 5.0 | 97 |
| 10.0 | 98 |
| 15.0 | 95 |
| 20.0 | 91 |
| 25.0 | 87 |
| 30.0 | 83 |
| 35.0 | 76 |
| 40.0 | 71 |
| 45.0 | 63 |
| 50.0 | 55 |

As shown in Table 6, the conversion rate is greater than 90% for a concentration of substrate of 5% to 20%, and the greatest conversion rate is 98% for a concentration of substrate of 10%.

### Experimental Example 5

### Identification of Kaempferol-3-O-Rutinoside

The products prepared in Examples 1 and 2 and Experimental Examples 1 to 4 exhibited the following characteristics, and were thereby identified as kaempferol-3-O-rutinoside (Varian Gemini 2000 300 MHz, Varian Company).

<Physicochemical properties of kaempferol-3-O-rutinoside>

Property: light greenish yellow micro crystal

Positive FAB-MS: 595[M+H]+

¹H-NMR: 6.31 (1H, d, 2, H6), 6.63 (1H, d, 2, H8), 7.03 (2H, d, 8, H3′, 5′), 8.21 (2H, d, 8, H2′, 6′), 12.04 (1H, s, 5-OH), 1.10 (314, d, 4, Me-rha), 4.61 (1H, br s, H1-rha), 5.20 (1H, d, 8, H1-glc)

¹³C-NMR: 156.6, 133.5, 177.5, 161.3, 98.9, 164.2, 93.8, 156.9, 104.2, 121.1, 130.9, 115.2, 159.9, 115.2, 130.9, 101.6, 74.4, 76.7, 70.9, 76.0, 67.1, 100.8, 70.5, 70.2, 72.1, 68.3, 17.7

### Example 3

### Preparation of Kaempferol-3-O-Rutinoside by Enzyme Reaction of Camelliaside A

10 g of the extract of green tea seed obtained from Preparation Example 1 was dissolved in 100 ml of 0.1M acetic acid buffer solution (pH 4.5). 1.5 g of enzyme was added to the mixture, and the resultant mixture was reacted in a water bath at 37° C. for 48 to 75 hours. The corresponding enzymes are β-glucosidase (Example 3-1), cellulase-A (Example 3-2) and β-galactosidase (Example 3-3), and kaempferol-3-O-rutinoside is prepared using each enzyme. Thereafter, a removal rate of substrate is checked with a thin layer chromatography. When the substrate is completely removed, the mixture is heated in hot water (80˜100° C.) for 5-15 minutes to terminate the reaction. The reaction solution was concentrated under reduced pressure to remove the solvent, and the residues were added to ethanol and stirred for one to five rotations. The resulting precipitates were removed through filtration and the filtrate was then concentrated under reduced pressure to obtain crude products. The obtained crude products were purified with silica gel column chromatography (chloroform: methanol=8:14:1), thereby obtaining pure kaempferol-3-O-rutinoside.

### Example 4

### Preparation of Kaempferol-3-O-Rutinoside by Enzyme Reaction of Camelliaside B

The kaempferol-3-O-rutinoside is prepared from camelliaside B according to the same methods as those of Example 3, and the enzymes used are β-xylosidase (Example 4-1), xylanase (Example 4-2) and naringinase (Example 4-3).

### Experimental Example 6

### Measurement of Inhibitory Effect of Collagenase Expression

The effect of inhibiting collagenase expression achieved by kaempferol-3-O-rutinoside obtained from Examples 3 and 4 is measured and compared with that achieved by tocopherol and EGCG. Tocopherol and EGCG are anti-oxidation materials that regenerate an epidermal cell of skin and prevent skin aging.

Human fibroblasts were inserted in a 96-well microtiter plate containing the DMEM (Dulbecco's Modified Eagle's Media) media with 2.5% fetal bovine serum to a quantity of 5000 cells/well and cultured until the human fibroblasts grow by 90%, then cultured again in the serum-free DMEM media for 24 hours. The kaempferol-3-O-rutinoside of Examples 3 and 4, tocopherol and EGCG dissolved in the serum-free DMEM media were treated at a concentration of 1×10⁴M for 24 hours, and the cell culture media was harvested.

The degree of collagenase generation of harvested cell culture media was measured using an instrument for measuring collagenase (Amershampharmacia Company). The harvested cell culture media was added in a 96-well plate coated uniformly with first collagenase antibody, and antigen-antibody reaction was conducted for 3 hours in a thermostat.

After 3 hours, second collagen antibody bonded with chromophore was added in the 96-well plate to react for 15 minutes. After 15 minutes, color developing material was inserted at room temperature for 15 minutes to induce color development, then 1M sulfuric acid was added to terminate the reaction (color development). The color of the reaction solution is yellow and the degree of yellow color can differ according to a degree of reaction progression.

Absorbance of the 96-well plate having the yellow color was measured through an absorbency measurement at 405 nm and a degree of synthesis was calculated by the following equation 1. In the calculation, an absorbance of cell culture media untreated with the material was used as a control group. That is, a degree of collagenase expression of an untreated group is 100, and a degree of collagenase expression of a group treated with the material as compared to that of the control group is obtained. The results are shown in Table 7.

$$Degree of collagenase expression ( \% ) = \frac{\begin{matrix}Absorbance of cell group \\ treated with the material\end{matrix} }{Absorbance of control group} \times 100 \qquad [ Equation 1 ]$$

TABLE 7

| | | | Degree of |
|---|---|---|---|
| | | | collagenase |
| Materials | | | expression (%) |
| Untreated group | | | 100 |
| Tocopherol | | | 75 |
| EGCG | | | 60 |
| Example 3 | Example 3-1 | Kaempferol-3-O-rutinoside prepared from | 70 |
| | | camelliaside A using β-glucosidase (Almond) as a | |
| | | reaction enzyme | |
| | Example 3-2 | Kaempferol-3-O-rutinoside prepared from | 71 |
| | | camelliaside A using cellulase-A (Aspergillus | |
| | | niger) as a reaction enzyme | |
| | Example 3-3 | Kaempferol-3-O-rutinoside prepared from | 63 |
| | | camelliaside A using β-galactosidase (Aspergillus | |
| | | oryzae) as a reaction enzyme | |
| Example 4 | Example 4-1 | Kaempferol-3-O-rutinoside prepared from | 68 |
| | | camelliaside B using β- xylosidase (Aspergillus | |
| | | niger) as a reaction enzyme | |
| | Example 4-2 | Kaempferol-3-O-rutinoside prepared from | 73 |
| | | camelliaside B using xylanase (Trichoderma viride) | |
| | | as a reaction enzyme | |
| | Example 4-3 | Kaempferol-3-O-rutinoside prepared from | 70 |
| | | camelliaside B using naringinase (Penicillium | |
| | | decumbens) as a reaction enzyme | |

The lower the degree of collagenase expression, the greater the inhibiting effect of collagenase expression and the lower the occurrence of decomposition of collagen in skin. Therefore, the amount of generated wrinkle can be decreased. From the results of Table 7, it is confirmed that a degree of collagenase expression differs depending on the enzyme used in Examples 3 and 4, but that kaempferol-3-O-rutinoside of the present invention inhibits the collagenase expression in vitro. Also, the effect of inhibiting collagenase expression achieved by kaempferol-3-O-rutinoside is greater than that achieved by tocopherol, which is known as an anti-oxidation material.

### Experimental Example 7

### Measurement of Promoting Effect of Procollagen Generation

The promoting effect of procollagen generation achieved by kaempferol-3-O-rutinoside obtained from Examples 3 and 4 is measured and compared with that achieved by vitamin C. The procollagen is a material inducing collagen generation and is required to generate collagen and prevent aging. Vitamin C is known as an essential component for synthesis of collagen.

Human fibroblasts were inserted in a 96-well microtiter plate containing the DMEM (Dulbecco's Modified Eagle's Media) media with 2.5% fetal bovine serum to a quantity of 5000 cells/well and cultured until the human fibroblasts grow by 90%, then cultured again in the serum-free DMEM media for 24 hours. The kaempferol-3-O-rutinoside of Examples 3 and 4, and vitamin C dissolved in the serum-free DMEM media were treated at a concentration of 1×10⁻⁴M for 24 hours, and the cell culture media was harvested. After 24 hours, the amount of procollagen floating in the culture media was measured using procollagen type-1 C-peptide EIA kit (MK101, Takara, Japan).

In an untreated group, a degree of procollagen generation is 100. A degree of procollagen generation of a group treated with the material as compared to that of the control group is obtained. The results are shown in Table 8.

TABLE 8

| | | | Degree of |
|---|---|---|---|
| | | | procollagen |
| Materials | | | generation (%) |
| Untreated group | | | 100 |
| Vitamin C | | | 120 |
| Example 3 | Example 3-1 | Kaempferol-3-O-rutinoside prepared from | 121 |
| | | camelliaside A using β-glucosidase (Almond) as a | |
| | | reaction enzyme | |
| | Example 3-2 | Kaempferol-3-O-rutinoside prepared from | 118 |
| | | camelliaside A using cellulase-A (Aspergillus | |
| | | niger) as a reaction enzyme | |
| | Example 3-3 | Kaempferol-3-O-rutinoside prepared from | 138 |
| | | camelliaside A using β-galactosidase (Aspergillus | |
| | | oryzae) as a reaction enzyme | |
| Example 4 | Example 4-1 | Kaempferol-3-O-rutinoside prepared from | 145 |
| | | camelliaside B using β- xylosidase (Aspergillus | |
| | | niger) as a reaction enzyme | |
| | Example 4-2 | Kaempferol-3-O-rutinoside prepared from | 123 |
| | | camelliaside B using xylanase (Trichoderma viride) | |
| | | as a reaction enzyme | |
| | Example 4-3 | Kaempferol-3-O-rutinoside prepared from | 122 |
| | | camelliaside B using naringinase (Penicillium | |
| | | decumbens) as a reaction enzyme | |

The greater the degree of procollagen generation, the greater the degree of collagen generation. Therefore, generation of wrinkle can be prevented. From the results of Table 8, it is confirmed that the degree of procollagen generation differs depending on the enzyme used in Examples 3 and 4, but that kaempferol-3-O-rutinoside of the present invention promotes the procollagen generation in vitro. Also, the degree of procollagen generation achieved by kaempferol-3-O-rutinoside is substantially greater than that achieved by vitamin C, which is known as an essential component for synthesis of collagen.

### INDUSTRIAL APPLICABILITY

The present invention provides a method for the mass-production of kaempferol-3-O-rutinoside using an enzyme or microbe to remove the sugar selectively from kaempferol-3-O-rutinoside glycosides in a plant extract. The present invention further provides a composition of a skin external application containing the kaempferol-3-O-rutinoside for preventing wrinkle.

1. A method for preparing kaempferol-3-O-rutinoside represented in the following chemical formula 1 comprising isolating kaempferol-3-O-rutinoside from kaempferol-3-O-rutinoside glycosides through hydrolysis using one of an enzyme and a microbe that has the possibility of selective removal of a sugar.

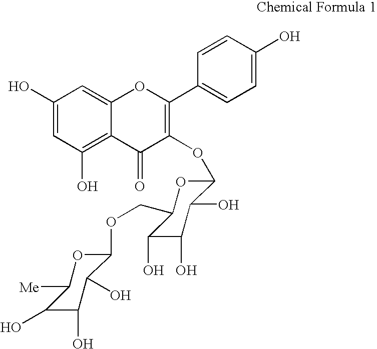

2. The method of claim 1, wherein the method for preparing kaempferol-3-O-rutinoside comprises:
(1) obtaining a plant extract containing kaempferol-3-O-rutinoside glycosides using one of water and organic solvent; and
(2) hydrolyzing the extract using one of an enzyme and a microbe to remove the sugar selectively from the kaempferol-3-O-rutinoside glycosides to isolate kaempferol-3-O-rutinoside.

3. The method of claim 1, wherein the kaempferol-3-O-rutinoside glycosides is camelliaside A or camelliaside B.

4. The method of claim 3, wherein the kaempferol-3-O-rutinoside is obtained by removing one of the sugar of galactopyranose group selectively from the said camelliaside A and the sugar of xylopyranose group selectively from the said camelliaside B.

5. The method of claim 4, wherein the enzyme to remove the sugar from camelliaside A is at least one selected from a group consisting of glucosidase, cellulase, galactosidase and amyloglucosidase, and the enzyme to remove the sugar from camelliaside B is at least one selected from a group consisting of xylosidase, xylanase and naringinase.

6. The method of claim 2, wherein the microbe is at least one selected from a group consisting of aspergillus sp., bacillus sp., penicillium sp., rhizopus sp., rhizomucor sp., talaromyces sp., bifidobacterium sp., mortierella sp., cryptococcus sp. and microbacterium sp.

7. The method of claim 1, wherein the reaction pH of the enzyme and the microbe is 4.0 to 5.5.

8. The method of claim 1, wherein the reaction temperature of the enzyme and the microbe is 30 to 50° C.

9. The method of claim 1, wherein the reaction time of the enzyme and the microbe is 48 to 76 hours.

10. The method of claim 2, wherein the plant extract containing kaempferol-3-O-rutinoside glycosides is the extract of green tea.

11. A composition of a skin external application comprising kaempferol-3-O-rutinoside represented in the following chemical formula 1 for preventing wrinkle

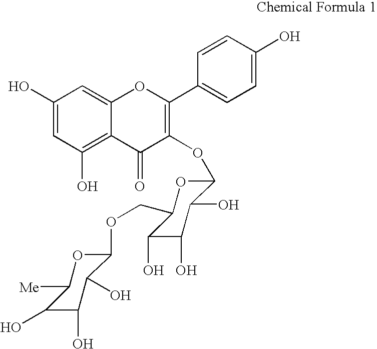

12. The composition of claim 11, wherein the kaempferol-3-O-rutinoside has combined synergetic action of the promotion of procollagen generation and the inhibitory activity of collagenase expression.

13. The composition of claim 11, wherein the kaempferol-3-O-rutinoside is contained in an amount of 0.0001% to 10% by weight based on the total weight of the composition.

14. The composition of claim 11, wherein the composition is a cosmetic composition formulated as one of a group consisting of skin softener, nutrition water, nutrition cream, massage cream, essence, eye cream, eye essence, cleansing cream, cleansing foam, cleansing water, pack, powder, body lotion, body cream, body oil, body essence, makeup base, foundation, hair-dyeing agent, shampoo, rinse and body washing agent.

15. The composition of claim 11, wherein the composition is a pharmaceutical composition formulated as one of a group consisting of ointment, gel, cream, patch and spraying agent.

16. The composition of claim 11, wherein the kaempferol-3-O-rutinoside is separated from green tea.
